(19) United States
(12) Patent Application Publication
(10) Pub. No.: US 2007/0142841 A1
(43) Pub. Date: Jun. 21, 2007

(54) SPINAL IMPLANT INSERTION INSTRUMENT AND METHOD

(76) Inventors: Cliff Reitzig, Riedheim-Weilheim (DE); Zoran Reiter, Emmingen (DE); Stephan Eckhof, Riedheim-Weilheim (DE)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 11/563,078

(22) Filed: Nov. 24, 2006

Related U.S. Application Data
(60) Provisional application No. 60/739,602, filed on Nov. 23, 2005.

(51) Int. Cl. A61F 2/46; A61B 17/90

(52) U.S. Cl. 606/90

(57) ABSTRACT

Insertion devices for positioning a spinal implant in an intervertebral disc space. The device distally advances an implant between flexible guide fingers.

### CROSS-REFERENCE TO RELATED APPLICATION

The subject matter of this application is related to the subject matter of U.S. Provisional Application Ser. No. 60/739,602, filed Nov. 23, 2005 and entitled “Spinal Implant Insertion Instrument and Method,” priority to which is claimed under 35 U.S.C. §119(e) and an entirety of which is incorporated herein by reference.

### BACKGROUND

The present invention relates to surgical methods and devices associated with implanting a spinal prosthesis into a spinal disc space.

The vertebrate spine is the axis of the skeleton on which all of the body parts “hang.” In humans, the normal spine has seven cervical, twelve thoracic and five lumbar segments. The lumbar spine sits upon the sacrum, which then attaches to the pelvis, and in turn, is supported by the hip and leg bones. The bony vertebral bodies of the spine are separated by intervertebral discs, which act as joints but allow known degrees of flexion, extension, lateral bending, and axial rotation.

The typical vertebra has a thick anterior bone mass called the vertebral body, with a neural (vertebral) arch that arises from the posterior surface of the vertebral body. The centra of adjacent vertebrae are supported by intervertebral discs. Each neural arch combines with the posterior surface of the vertebral body and encloses a vertebral foramen. The vertebral foramina of adjacent vertebrae are aligned to form a vertebral canal, through which the spinal sac, cord and nerve rootlets pass. The portion of the neural arch which extends posteriorly and acts to protect the spinal cord's posterior side is known as the lamina. The spinous process projects from the posterior region of the neural arch.

The intervertebral disc primarily serves as a mechanical cushion, permitting controlled motion between vertebral segments of the axial skeleton. The normal disc is a unique, mixed structure, comprised of three component tissues: the nucleus pulpous (“nucleus”), the annulus fibrosus (“annulus”) and two vertebral end plates. The two vertebral end plates are composed of thin cartilage overlying a thin layer of hard, cortical bone which attaches to the spongy, richly vascular, cancellous bone of the vertebral body. The end plates thus act to attach adjacent vertebrae to the disc. In other words, a transitional zone is created by the end plates between the malleable disc and the bony vertebrae.

The annulus of the disc is a tough, outer fibrous ring which binds together adjacent vertebrae. The fibrous portion, which is much like a laminated automobile tire, measures about 10 to 15 millimeters in height and about 15 to 20 millimeters in thickness. The fibers of the annulus consist of fifteen to twenty overlapping multiple plies, and are inserted into the superior and inferior vertebral bodies at roughly a 40-degree angle in both directions. This configuration particularly resists torsion, as about half of the angulated fibers will tighten when the vertebrae rotates in either direction, relative to each other. The laminated plies are less firmly attached to each other.

Immersed within the annulus, positioned much like the liquid core of a golf ball, is the nucleus. The healthy nucleus is largely a gel-like substance having a high water content, and like air in a tire, serves to keep the annulus tight yet flexible. The nucleus-gel moves slightly within the annulus when force is exerted on the adjacent vertebrae while bending, lifting, etc.

The spinal disc may be displaced or damaged due to trauma or a disease process. A disc herniation occurs when the annulus fibers are weakened or torn and the inner tissue of the nucleus becomes permanently bulged, distended, or extruded out of its normal, internal annulus confines. The mass of a herniated or “slipped” nucleus tissue can compress a spinal nerve, resulting in leg pain, loss of muscle control, or even paralysis. Alternatively, with discal degeneration, the nucleus loses its water binding ability and deflates, as though the air had been let out of a tire. Subsequently, the height of the nucleus decreases causing the annulus to buckle in areas where the laminated plies are loosely bonded. As these overlapping laminated plies of the annulus begin to buckle and separate, either circumferential or radial anular tears may occur, which may contribute to persistent and disabling back pain. Adjacent, ancillary spinal facet joints will also be forced into an overriding position, which may create additional back pain.

Whenever the nucleus tissue is herniated or removed by surgery, the disc space will narrow and may lose much of its normal stability. In many cases, to alleviate back pain from degenerated or herniated discs, the nucleus is removed and the two adjacent vertebrae are surgically fused together. While this treatment alleviates the pain, all discal motion is lost in the fused segment. Ultimately, this procedure places a greater stress on the discs adjacent to the fused segment as they compensate for lack of motion, perhaps leading to premature degeneration of those adjacent discs.

One surgical concern is the potential damage imparted upon the annulus during implantation surgery. The normal annular plies act to keep the annulus tight about the nucleus. During surgery, a surgical knife or tool is used to completely sever some portion of the annulus and/or remove an entire section or a “plug” of the annulus tissue. The size of such a plug is often determined according to the space requirements of a particular measurement tool used to estimate the size of the intervertebral space or the space required by of an implantation tool utilized to insert a prosthetic disc into the intervertebral space. When an entire section of the annulus is cut or removed to insert the prosthetic device, the layers making up the annulus “flay” and/or “pull back” and the constraining or tightening ability of that portion of the annulus is lost. Further, the chances of the annulus healing with restoration of full strength are greatly diminished, while the likelihood of nucleus reherniation is increased. An even greater concern arises where a significant portion of the annulus is removed entirely. A more desirable solution is to leave as much of the annulus intact as possible during and after implantation.

In light of the above, smaller prosthetic nucleus bodies have been developed. With the reduction in prosthetic size, the ability to leave portions of the annulus intact during and after implantation has been at least partially realized. In conjunction with such prostheses, potential improvements reside in insertion devices associated with the implantation of such prostheses.

### SUMMARY

Some aspects in according with principles of the present disclosure relate to an insertion device for implanting a spinal implant in an intervertebral disc space. The insertion device includes a guide piece, an implant keeper, a guide structure, and a handle assembly. The guide piece releasably receives the spinal implant and, in turn, is coaxially received in the implant keeper such that both the spinal implant and the guide piece can be slidably disposed within the implant keeper. The guide structure extends distal the implant keeper. Such guide structures can include flexible members configured to flexibly guide the spinal implant through an annulus hole. The handle assembly is configured to effectuate distal movement of the guide piece relative to the implant keeper to deliver the guide piece and the implant from the implant keeper into the intervertebral space.

Yet other aspects of the present disclosure relates to a method of inserting a spinal implant into an intervertebral disc space through an annulus. The method includes providing an insertion device such as those described in relation to other aspects of the present invention. The method also includes forming a hole in a disc annulus and removing a portion of a disc nucleus. A portion of a the guide structure is inserted into the hole. The implant is delivered along the guide structure and into the intervertebral space by actuating the handle assembly. In some embodiments, the guide structure includes opposing fingers that effectuate distraction of adjacent vertebrae with distal movement of the spinal implant therebetween.

### BRIEF DESCRIPTION OF THE DRAWINGS

FIG. 1 illustrates an embodiment insertion device in accordance with principles of the invention.

FIG. 2 is an exploded, perspective view of the embodiment insertion device of FIG. 1.

FIG. 3 is a front, cross-sectional view of a guide piece along a central longitudinal axis of the embodiment insertion device of FIG. 1.

FIG. 4 is a front, cross-sectional view of an implant keeper along the central longitudinal axis of the embodiment insertion device of FIG. 1.

FIG. 5 is a front, cross-sectional view of a transition assembly along the central longitudinal axis of the embodiment insertion device of FIG. 1.

FIG. 6 is a front, cross-sectional view of an insertion shaft along the central longitudinal axis of the embodiment insertion device of FIG. 1.

FIG. 7 is a front, cross-sectional view of a push rod along the central longitudinal axis of the embodiment insertion device of FIG. 1.

FIG. 8 is a front, cross-sectional view of a grip member along the central longitudinal axis of the embodiment insertion device of FIG. 1.

FIG. 9 is a front, cross-sectional view of a cap along the central longitudinal axis of the embodiment insertion device of FIG. 1.

FIG. 10 is a front, cross-sectional view of the embodiment insertion device of FIG. 1 along the central longitudinal axis.

FIG. 11A is a top view of the embodiment insertion device of FIG. 1.

FIG. 11B is side view of the embodiment insertion device of FIG. 1.

FIG. 11C is a front view of the embodiment insertion device of FIG. 1.

FIGS. 12A-12C are front views illustrating an embodiment method of inserting a spinal implant in accordance with principles of the invention.

FIG. 13 is a perspective view of another embodiment implant keeper in accordance with principles of the invention.

FIG. 14A-14C illustrate a portion of an alternative embodiment insertion device.

### DETAILED DESCRIPTION

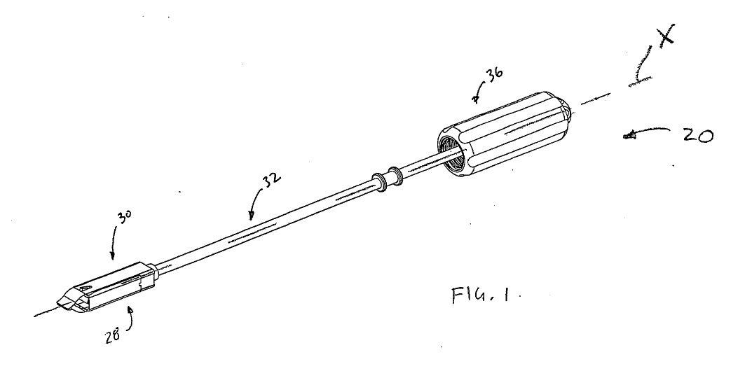

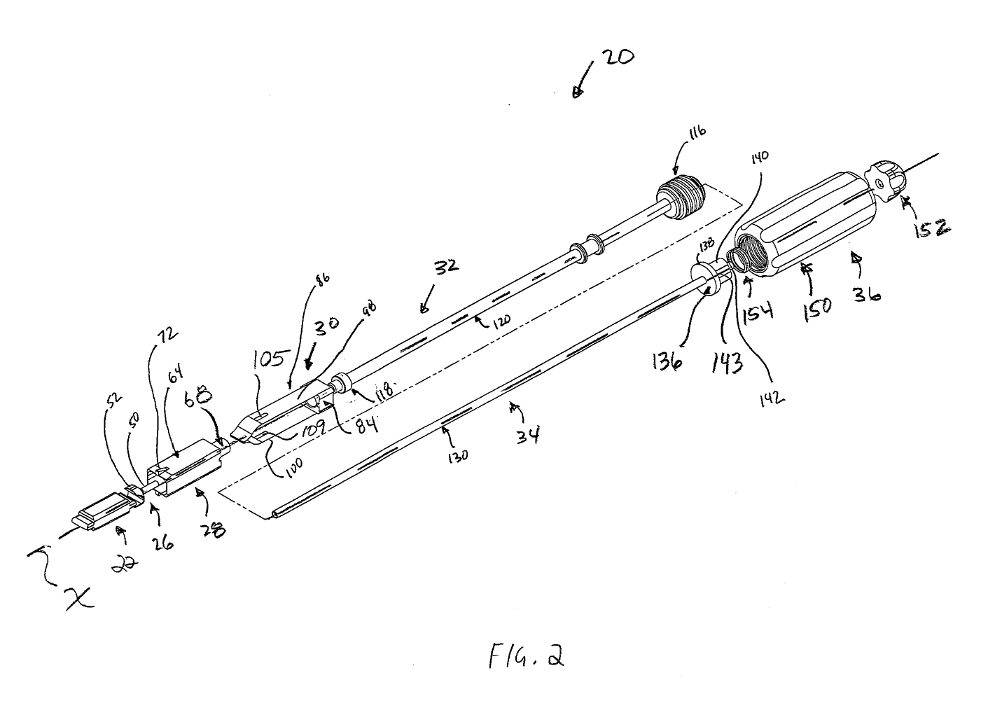

One embodiment of an insertion device 20 in accordance with principles of the present disclosure is shown in an assembled form in FIG. 1. With additional reference to FIG. 2, the insertion device 20 is generally used to position a spinal implant 22 in an intervertebral space (not shown). As a point of reference, the implant 22 is illustrated generically in FIG. 2, and can assume a wide variety of forms (e.g., a prosthetic spinal disc nucleus device, such as devices available from Raymedica of Bloomington, Minn. under the tradenames PDN®, PDN-SOLO®, PDN-SOLO XL™. and Hydraflex™). Regardless, in terms of form, the insertion device 20 defines a central longitudinal axis X and in some embodiments includes a guide piece 26, an implant keeper 28, a transition assembly 30, an insertion shaft 32, a push rod 34, and a handle assembly 36. The insertion device 20, and its component parts, can be formed of surgically safe materials, including polymeric and/or metallic materials. In general relational terms, the guide piece 26 releasably receives, or otherwise releasably engages, the implant 22. Both the implant 22 and the guide piece 26 are slidably received within the implant keeper 28 prior to delivering the implant 22 into the disc space. The implant keeper 28 is connected to the insertion shaft 32 via the transition assembly 30. The push rod 34 is coaxially received within the insertion shaft 32 and interfaces with the handle assembly 36 such that rotation of the handle assembly 30 rotates and displaces the push rod 34 in a distal direction, which, in turn, acts to distally displace the guide piece 26 and the spinal implant 22 from the implant keeper 28.

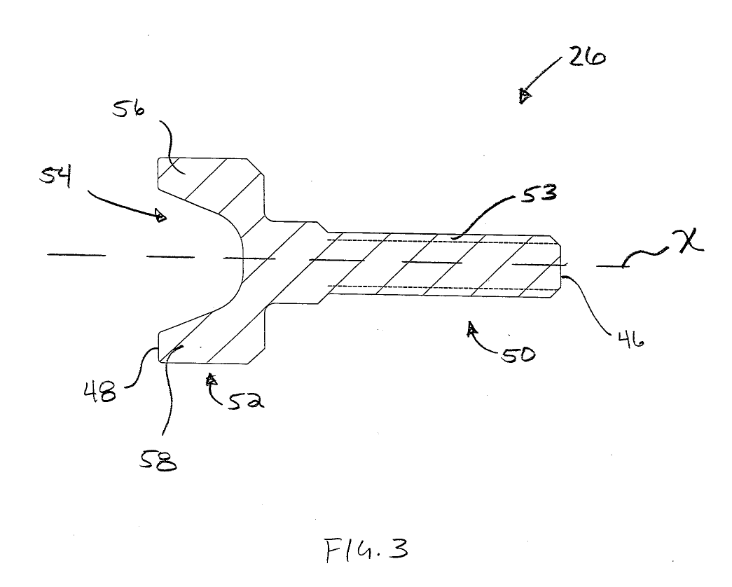

With additional reference to FIG. 3, the guide piece 26 extends from a proximal end 46 to a distal end 48. In some embodiments, the guide piece 26 defines an outer profile transverse to the central longitudinal axis X that is equal to or less than that of the spinal implant 22 (FIG. 2). As will be understood in greater detail below, this feature facilitates insertion of the guide piece 26 into spaces otherwise sized to receive the implant 22. To this end, minimizing an outer size or profile of the guide piece 26 can promote a more optimally sized hole (e.g., having a minimum transverse outer diameter) in a disc annulus (not shown) in order to insert the implant 22/guide piece 26. However, other embodiments of the present disclosure include the guide piece 26 defining a greater transverse outer diameter than the implant 22, and may also assist guiding the implant 22 into the intervertebral disc space.

In one embodiment, the guide piece 26 includes a base 50 defining the proximal end 46, and a receptacle 52 extending from the base 50 to the distal end 48. The base 50 is cylindrical and is configured to be coaxially inserted into a portion of the implant keeper 28. Additionally, as will be described in greater detail below, the base 50 is configured to be secured to the push rod 34. Along these lines, the base 50 includes or forms a threaded surface 53 (designated generally by dotted lines in FIG. 3) configured to mate with a corresponding feature of the push rod 34.

The receptacle 52 forms or defines a cavity 54 configured to selectively receive at least a portion the implant 22. In particular, the cavity 54 is generally sized and shaped in accordance with a size and shape of the implant 22, for example commensurate with an end of the implant 22. Thus, while the cavity 54 is illustrated has having curvilinear shape, a wide variety of other shapes are also acceptable, and can be selected in accordance with an exterior form or footprint of the implant 22 in question. In order to facilitating pushing of the implant 22, the receptacle 52 can include two distinct protrusions, a top leaflet 56 and a bottom leaflet 58. The leaflets 56, 58 oppose one another and define opposing sides of the cavity 54. In some embodiments, the leaflets 56, 58 are substantially rigid. Alternatively, one or both of the leaflets 56, 58 can exhibit some flexibility such that they can deflect inwardly or can splay, or deflect, outwardly relative to the central longitudinal axis X. Regardless, the spinal implant 22 can be generally maintained between the two leaflets 56, 58 while still being removable therefrom.

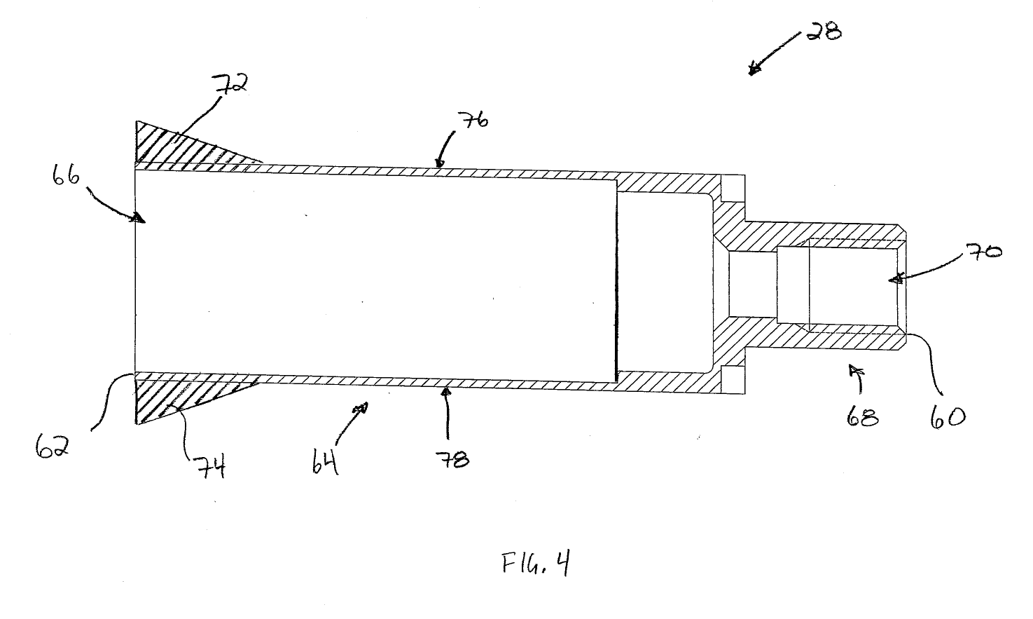

With reference to FIGS. 2 and 4, the implant keeper 28 defines a proximal end 60 and a distal end 62. The implant keeper 28 can be akin to an open-ended box and includes a receptacle portion 64 extending from the distal end 62, the receptacle portion 64 defining a cavity 66. The cavity 66 can be defined by the implant keeper 28 to have a variety of shapes corresponding at least generally with a shape of the spinal implant 22 (e.g., box-shaped, cylindrical, etc.). Regardless, the cavity 66 is exteriorly open relative to the implant keeper 28 at the distal end for receiving the spinal implant 22 (it being understood that at an end opposite the distal end 62, an exterior opening, if any, to the cavity 66 can be smaller than the spinal implant 22). The implant keeper 28 also includes a base 68 having an inner lumen 70 extending from the receptacle portion 64 to the proximal end 60. In some embodiments, for example with embodiments in which the implant 22 includes a hydrogel core (not shown), the implant keeper 28 also acts to maintain the spinal implant 22 in a smaller size and/or in a particular shape prior to implantation of the spinal implant 22. Exemplary teachings of this principle can be found in U.S. Pat. No. 6,533,817, the teachings of which are incorporated herein by reference.

The receptacle portion 64, and in particular the cavity 66, is configured to coaxially receive the guide piece 26 and the spinal implant 22. With this in mind, the cavity 66 can define an internal distal taper. In some embodiments, the taper serves to prevent inadvertent ejection of the implant 22 from the cavity 66 in that the implant 22 can be more robustly secured to the receptacle portion 64 in a region of the tapered diameter. Regardless, the receptacle portion 64 includes a top protrusion 72 and a bottom protrusion 74 configured to mate with a portion of the transition assembly 30 (as described below) and defines a top face 76 and a bottom face 78. The top protrusion 72 is a generally angular member that extends from the top face 76 in a lengthwise direction, expanding distally in height and terminating at or adjacent the distal end 62. The bottom protrusion 74 can be essentially identical to the top protrusion 72, but formed as a projection from the bottom face 78. The top face 76 and the bottom face 78 can also define a distal taper in an outer profile of the receptacle portion 64 to facilitate insertion of the distal end 62 during use.

As will be described in greater detail below, the top and bottom protrusions 72, 74 are adapted to assist with positioning the implant keeper 28 at a desired depth within or relative to the intervertebral disc space. As such, the top and bottom protrusions 72, 74 are flush with proximal end 62. In other embodiments, the top and bottom protrusions 72, 74 are recessed proximally from the distal end 62 to facilitate deeper insertion of the implant keeper 28 within the intervertebral disc space. Alternatively, one or both of the top and bottom protrusions 72, 74 can be eliminated.

The base 68 has a generally cylindrical shape, with the inner lumen 70 being sized to coaxially and slidably receive the push rod 34. In this regard, the inner lumen 70 extends through an entirety of the base 68 and is open to the cavity 66. Furthermore, the base 68 is also configured to be coaxially received in the transition assembly 30, as will be described in greater detail below. To this end, the base 68 can form exterior threads (not shown) for mating with a corresponding feature the insertion shaft 32.

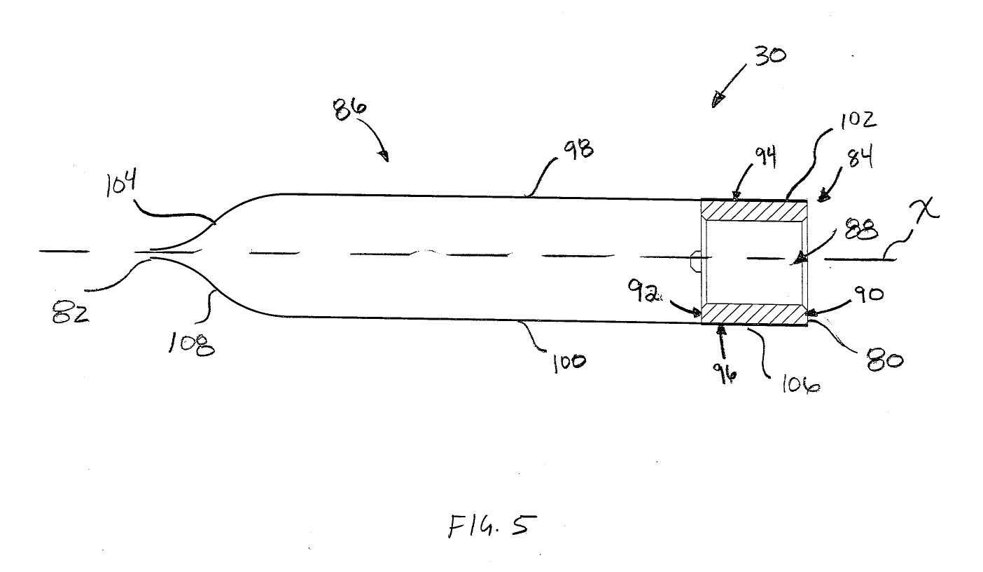

With reference to FIGS. 2 and 5, the transition assembly 30 defines a proximal end 80 and a distal end 82, and includes a base 84 and a guide structure or assembly 86. The base 84 defines and extends from the proximal end 80, and can be generally box-shaped, or rectangular, forming an inner lumen 88 extending lengthwise through an entirety of the base 84. The lumen 88 is sized to coaxially receive the base 68 of the implant keeper 28. Finally, the base 84 can be described as defining a proximal face 90, a distal face 92, a top face 94, and a bottom face 96.

The guide structure 86 extends distally from the base 84 and includes, in some embodiments, a first guide finger 98 secured to the top face 94 of the base 84 and a second guide finger 100 secured to the bottom face 96. The guide fingers 98, 100 combine to define the distal end 82.

The first guide finger 98 extends from a proximal portion 102 to a distal portion 104 to define an overall length. The proximal portion 102 is adapted to be mounted to the top face 94 of the base 84. The distal portion 104 defines a recurved shape as shown. The guide finger 98 can also include a notch 105 (FIG. 2) configured to receive the top protrusion 72 associated with the implant keeper 28. This relationship can serve at least two functions. First, it helps retain the transition assembly 30 on the implant keeper 28 when the first guide finger 98 is in a non-deflected state. Second, and as will be described in greater detail below, the protrusion 72 can protrude through the notch 105 and be abutted against an annulus or other vertebral structure to allow a user to “feel” when the device 20 has been properly positioned.

The second guide finger 100 defines a proximal portion 106 and a distal portion 108, the proximal portion 106 secured to the bottom face 96 of the base 84. The distal portion 108 also defines a recurved shape. The second guide finger 100 also includes a notch 109 (FIG. 2) configured to receive the bottom protrusion 74 associated with the implant keeper 28. This relationship can serve similar functions to those described above in association with the notch 105 and protrusion 72.

In one embodiment, the first guide finger 98 and the second guide finger 100 are maintained in opposing positions by the base 84, and can be integrally formed with the base 84. In other embodiments, the guide fingers 98, 100 are separately formed from the base 84 and secured thereto via such means as adhesives or mechanical fasteners. The guide fingers 98, 100 are transversely flexible, defining a thin, generally elongate, and rectangular shape (e.g., metal “tape”). In this manner, the guide fingers 98, 100 can incorporate sufficient flexibility to deflect outwardly away from the central longitudinal axis X, and one another, when the spinal implant 22 is pressed between them as described in greater detail below (e.g., the distal portions 104, 108 can expand or deflect away from one another relative to the natural or relaxed state of FIG. 5, pivoting at the base 84). In addition, at least an interior face of each of the fingers 98, 100 (i.e., side of the respective finger 98 or 100 “facing” to other finger 98 or 100) is smooth so as to minimize or eliminate possible damage to the spinal implant 22 as the spinal implant 22 passes along/contacts the interior face.

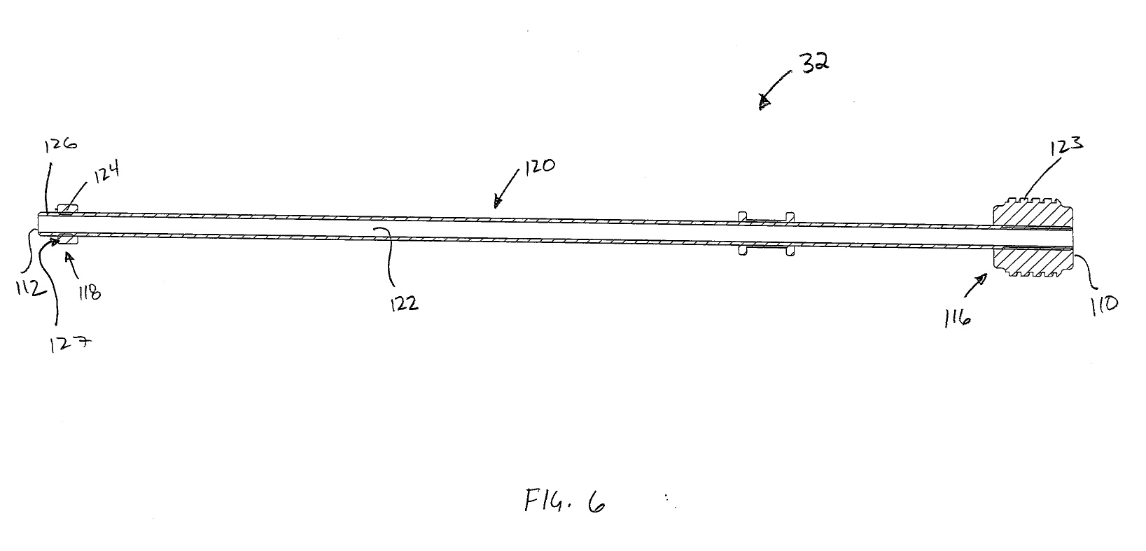

With reference to FIGS. 2 and 6, the insertion shaft 32 can define an elongate tubular shape, extending from a proximal end 110 to a distal end 112. Proximate the proximal end 110, the insertion shaft 32 includes a hub 116. Proximate the distal end 112, the insertion shaft 32 includes a mating head 118. A shaft body 120 extends between the hub 116 and the mating head 118. In one embodiment, an inner lumen 122 extends through an entirety of the hub 116, the mating head 118, and the body 120. The inner lumen 122 has a minimum diameter sized to receive the push rod 34.

The hub 116 is secured about the body 120 and defines an outer radius greater than that of the body 120. The hub 116 forms a threaded surface 123 configured to be threadably secured to a corresponding feature of the handle assembly 36 as described in greater detail below.

The mating head 118 includes a collar 124 and a distal portion 126 of the body 120. The collar 124 defines a greater outer diameter than that of the body 120 and is generally configured to abut against the proximal face 90 of the base 84 of the transition assembly 30. As described below, a distal face 127 of the collar 124 is adapted to form a substantially continuous contact with the proximal face 90 of the base 84 of the transition assembly 30. The distal portion 126 extends distally from the collar 124 and is configured for coaxial insertion into the base 68 of the implant keeper 28. More particularly, the mating head 118 facilitates a secure connection between the implant keeper 28, the transition assembly 30, and the insertion shaft 32. For example, the distal portion 126 can include or form threads (not shown) for threadably securing the insertion shaft 32 into the implant keeper 28 to secure the insertion shaft 32, the transition assembly 30, and the implant keeper 28 together.

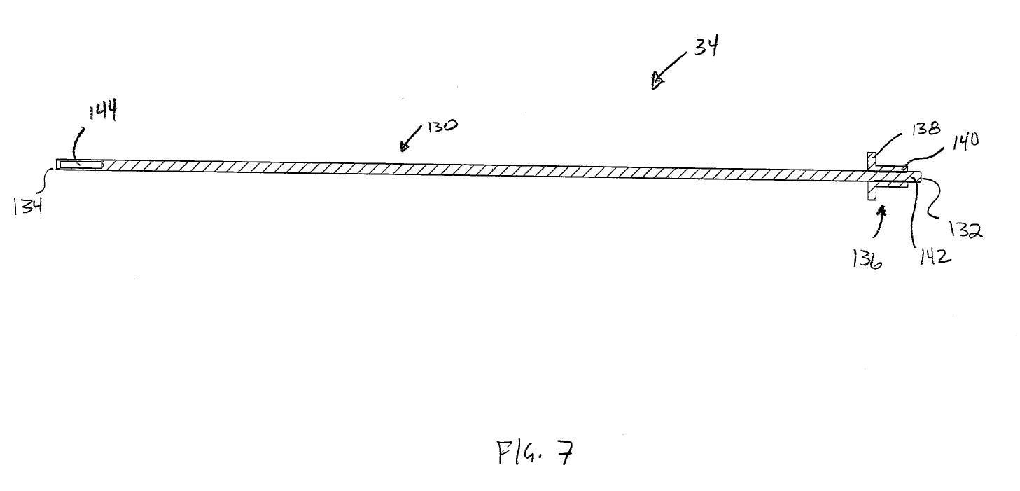

With reference to FIGS. 2 and 7, the push rod 34 includes a solid, elongate body 130 extending from a proximal end 132 to a distal end 134. The push rod 34 also includes a collar assembly 136 disposed adjacent the proximal end 132. In some embodiments, the body 130 is generally cylindrical in shape. Generally, the body 130 is configured to be coaxially received within the insertion shaft 32 and the implant keeper 28.

The collar assembly 136 includes a flange 138, a rim 140, and a shank 142 extending proximally from the rim 140. The flange 138 defines a greater diameter than the body 130 of the push rod 34 and can be generally circular, for example, in transverse cross-section. The rim 140 is substantially ovoid is transverse cross-section and defines a flat 143 (FIG. 2). As will be described in greater detail below, the flat 143 and/or ovoid cross-section result in the rim 140 being “keyed” to a portion of the handle assembly 36. The proximal portion 142 forms a threaded surface (not shown) configured to mate with a portion of the handle assembly 36. The shank 142 is effectively an extension of the body 130 proximal the rim 140, and thus can be provided as an integral feature of body 130.

An inner cavity 144 is formed by the body 130 at or adjacent the distal end 134. The inner cavity 144 can include an internal, threaded surface (not shown) configured to threadably engage a corresponding surface of the guide piece 26.

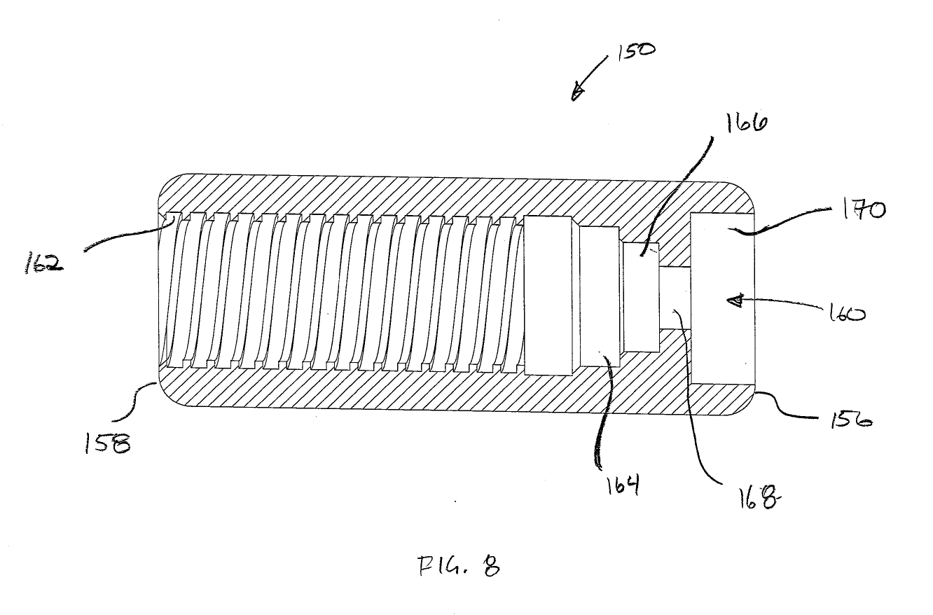

With reference to FIG. 2, one embodiment of the handle assembly 36 includes a grip member 150, a cap 152, and a spring 154. With additional reference to FIG. 8, the grip member 150 is generally tubular in shape and forms a passage 160 extending between a proximal end 156 to a distal end 158. In general terms, the inner lumen 160 is configured to coaxially receive corresponding features of the push rod 34 and the insertion shaft 32. To this end, the grip member 150 can include or form internal threads 162 along the inner lumen 160 proximate the distal end 158. The threads 162 are configured to receive and mate with a corresponding surface of the insertion shaft 32.

The inner lumen 160 can be stepped in diameter to define a collar seat 164, a spring seat 166, a keyed portion 168, and a cap receptacle 170. The collar seat 164 is configured to coaxially receive the flange 138 of the push rod collar assembly 136. In particular, the collar seat 164 acts as a stop, to arrest proximal displacement of the collar assembly 136 relative to the grip member 150 when the flange 138 comes into contact, or is stopped against the collar seat 164.

The spring seat 166 is configured to coaxially receive the spring 154 and act as a stop, to arrest proximal displacement of the spring 154 relative to the grip member 150.

The keyed portion 168 is configured to coaxially receive the rim 140 of the push rod collar 136. In particular, the keyed portion 168 includes a complementary shape to the flat 143 and/or the ovoid shape of the rim 140. In this manner, the keyed portion 168 acts as a stop to arrest rotation of the rim 140 (and therefore the push rod 34) relative to the grip member 150. However, the keyed portion 168 does not otherwise interfere with distal or proximal motion of the rim 140 relative to the grip member 150.

The cap receptacle 170 is similarly configured to coaxially receive the cap 152. The cap receptacle 170 acts to stop distal displacement of the cap 152 relative to the grip member 150 upon contacting the cap receptacle 170.

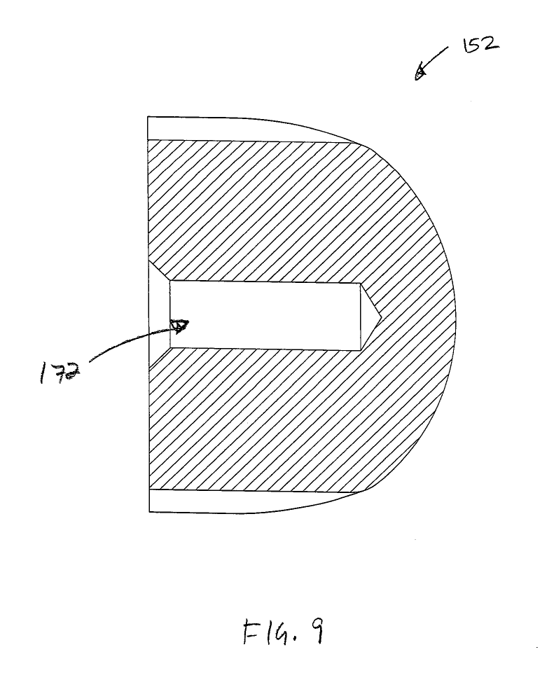

With reference to FIGS. 2 and 9, the cap 152 is dome-shaped and includes an inner lumen 172 sized to receive the shank 142 of the collar assembly 136. For example, the inner lumen 172 can form a threaded surface (not shown) configured to threadably mate with threads of the shank 142. In this manner, the cap 152 can be secured to the shank 142, and, in turn, the push rod 34.

With specific reference to FIG. 2, the spring 154 can take a variety of forms and is configured to bias the push rod 34 distally relative to the grip member 150 upon final assembly.

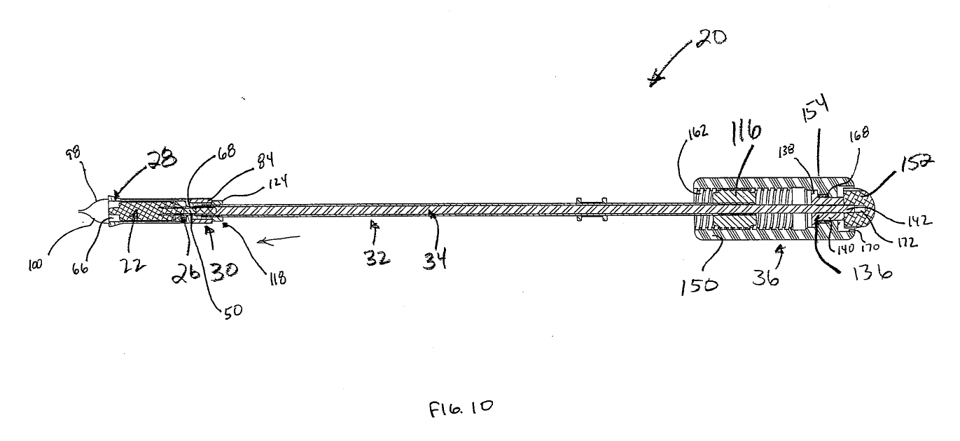

In light of the above description, and with general reference to FIGS. 2 and 10, one embodiment assembly of the insertion device 20 in accordance with the present disclosure can be described. In general terms, each of the spinal implant 22, the guide piece 26, the implant keeper 28, the transition assembly 30, the insertion shaft 32, the push rod 34, and the handle assembly 36 are coaxially aligned relative to one another along the central longitudinal axis X of the insertion device 20.

The spinal implant 22 is coaxially and slidably received within the cavity 54 (FIG. 3) of the guide piece 26 between the top leaflet 56 (FIG. 3) and the bottom leaflet 58 (FIG. 3). In turn, the implant 22, as well as the guide piece 26, is slidably and coaxially received within the cavity 66 of the implant keeper 28. The implant keeper 28, and in particular the base 68, is coaxially received and secured within the base 84 of the transition assembly 30.

With the transition assembly 30 and the implant keeper 28 so-assembled, the first guide finger 98 and the second guide finger 100 extend distally over a top face 76 (FIG. 4) and the bottom face 78 (FIG. 4) of the implant keeper 28, respectively. The fingers 98, 100 smoothly recurve toward one another distal the implant keeper 28.

The collar 124 of the insertion shaft mating head 118 is abutted and secured against the base 68 of the implant keeper and the base 84 of the transition assembly 30. Additionally, the distal portion 126 (FIG. 6) is coaxially received by and secured to the base 68 and the base 84. For example, the distal portion 126 can be threadably secured the base 68.

As alluded to above, the hub 116 of the insertion shaft 32 is configured to be coaxially received in the inner lumen 160 (FIG. 8) of the grip member 150. In particular, the threaded surface 123 (FIG. 6) of the hub 116 threadably engages the threads 162 of the grip member 150 such that relative rotation between the grip member 150 and the insertion shaft 32 induces distal and/or proximal movement of the grip member 150 relative to the insertion shaft 32 via a screw-type arrangement.

The push rod 34 is coaxially received within the grip member 150, the insertion shaft 32, and the implant keeper 28. As will be described in greater detail below, the push rod 34 is also configured to be secured to in the base 50 (FIG. 3) of the guide piece 26 and is threadably secured thereto.

The spring 154 is also coaxially disposed in the grip member 150. In particular, the spring 154 is coaxially received and seated in the spring seat 166 (FIG. 8). In turn, the rim 140 of the push rod 34 is coaxially received in the spring 154 and the keyed portion 168 of the grip member 150. In turn, the flange 138 is slidably and coaxially received and seated in the collar seat 164 (FIG. 8). The shank 142 extends proximally into the cap receptacle 170. In this manner, the cap 152 can be secured to the shank 142. In particular, the inner lumen 172 of the cap 152 coaxially receives, and is threaded onto, the shank 142 to secure the cap 152 to the push rod 34. Thus, the cap 152 is coaxially and slidably received in the cap receptacle 170 of the grip member 150.

With reference to FIG. 10 in particular, an interaction between the assembled insertion shaft 32, push rod 34, grip member 150, cap 152, and spring 154 can be described. With this assembly, the spring 154 biases the flange 138 of the collar assembly 136 (and therefore the push rod 34) in a distal direction relative to the grip member 150. However, resistance at the distal end 134 (FIG. 7) of the push rod 34 and/or contact between the cap 152 and the cap receptacle 170 stops distal displacement of the push rod 34 relative to the grip member 150. In turn, proximal displacement of the push rod 34 relative to the grip member 150 is arrested when the flange 138 is seated against the collar seat 164 (FIG. 8). In other words, with sufficient force or resistance pushing in the proximal direction, the spring 154 is deflected such that the collar assembly 136 is at a “hard stop” against the collar seat 164. During assembly, the spring 154 allows for sufficient engagement of the push rod 34 with the guide piece 26 prior to the grip member 150 engaging base 116 of insertion shaft 32.

The rim 140 of the collar assembly 136 is seated in the keyed portion 166 (FIG. 6) of the grip member 150 such that the rim 140 is not free to rotate, but can slide distally or proximally according to the relationships just described. In this manner, rotation of the grip member 150 is translated into rotation of the push rod 34.

With the hub 116 of the insertion shaft 32 threaded into the grip member 150, rotation of the grip member 150 in a first direction results in distal movement of the grip member 150 relative to the insertion shaft 32. Rotation of the grip member 150 in a second, opposite direction results in proximal motion of the grip member 150 relative to the insertion shaft 32. Additionally, as described above, distal and/or proximal motion, as well as rotation, of the grip member 150 is translated to the push rod 34. As such, rotation of grip member 150 results in distal or proximal actuation of the push rod 34, as well as rotation of the push rod 34, relative to the insertion shaft 32.

The distal movement of the push rod 34 eventually results in the distal end 134 (FIG. 7) of the push rod 34 abutting against the base 50 of the guide piece 26. In operation, the spring 154 and cap 152 interaction with the push rod 34 in the grip member 150 provides some “play” to assist in aligning the threads of the cavity 144 (FIG. 7) with the threads at the base 50 of the guide piece 26.

With threaded engagement between the push rod 34 and the guide piece 26, the grip member 150 continues to rotate and advance the push rod 34 distally. The thread spacing and/or pitch on the base 116 of the insertion shaft 32 and the grip member 150 is larger than that of the guide piece 26 and push rod 34. In this manner, the push rod 34 simultaneously advances the guide piece 26 distally and is “screwed onto” the base 50 of the guide piece 26. As the push rod 34 is advancing the guide piece 26 and being secured thereto, the implant 22 is pushed distally within the implant keeper 28. In particular, the distal movement of the guide piece 26 is translated to the implant 22, which, in turn, slides distally within the implant keeper 28.

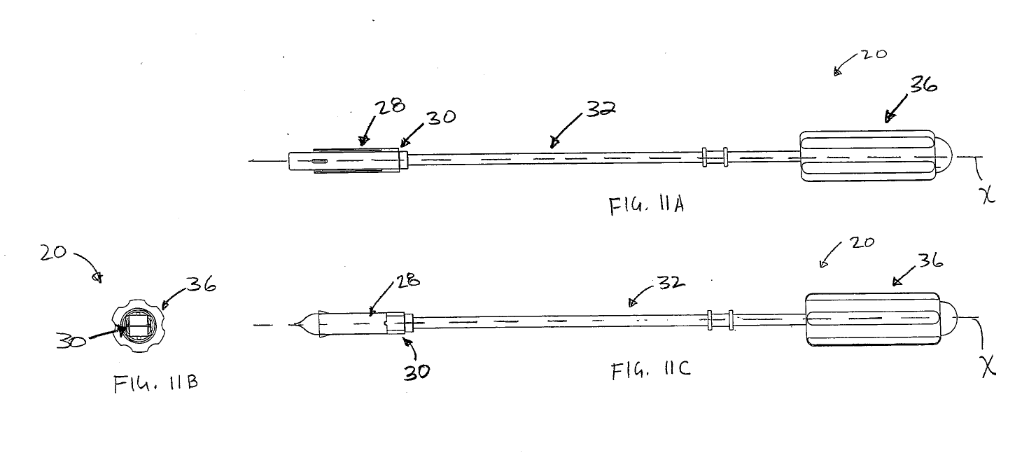

FIGS. 11A-11C show the assembled device 20 from a variety of views for additional reference.

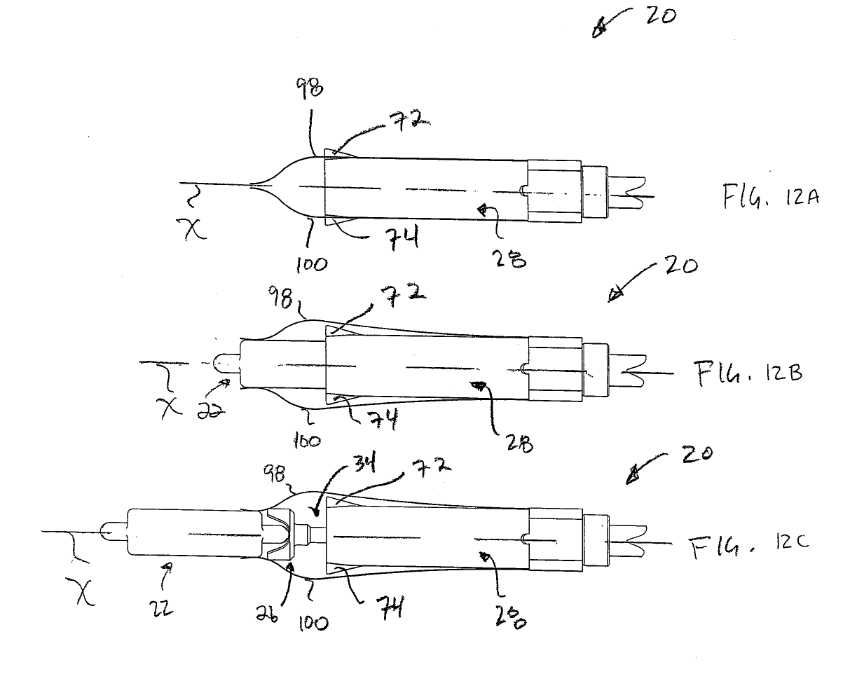

During use, and with reference to FIGS. 12A-12C, the implant 22 is eventually forced from the implant keeper 28 and comes into contact with the guide fingers 98, 100. In turn, the guide fingers 98, 100 deflect outwardly away from the central longitudinal axis X as the implant 22 is forced therebetween. Finally, the implant 22 is forced distal to the guide fingers 98, 100 and is freed from the guide piece 26. At the point the implant 22 is free of the guide wings 98, 100, the push rod 34 has been threaded onto the guide piece 26 such that the two are secured together.

In light of the above, one embodiment method of inserting the spinal implant 22 into a disc space (not shown) in accordance with the present invention can be described as follows with reference to FIGS. 12A-12C. For reference, a disc space maintains a disc nucleus and is bounded, and defined by, a top vertebra, a bottom vertebra, and a disc annulus. An opening is formed in the disc annulus and a portion of the disc nucleus is removed. The insertion device 20, and in particular the guide fingers 98, 100, are advanced toward the intervertebral space, for example toward the disc annulus opening. The device 20 can be advanced, for example, by grasping and manipulating the grip member 150 (FIG. 8).

As the distally protruding guide fingers 98, 100 come into contact with the top and bottom vertebrae, respectively, they can help guide and center the implant keeper 28 relative to the annulus opening. As the guide fingers 98, 100 and implant keeper 28 are advanced distally, the top and bottom protrusions 72, 74 of the implant keeper are brought into contact with the annulus and/or vertebrae surrounding the annulus opening, alerting a user that the insertion device 20 has been sufficiently distally advanced to a desired position. For example, the user can tactilely sense such contact resistance to movement. In other embodiments, the protrusions 72, 74, and/or other portions of the device 10 can include radiopaque materials and/or coatings such that the device 10 can be advanced under x-ray. Once the device 10 has been advanced to the desired position, the guide fingers 98, 100 extend through the annulus opening and partially into the intervertebral space. In connection with this distal advancement/movement, the wedge-like arrangement of the guide fingers 98, 100 (distal the implant keeper 28) serves to distract the opposing vertebrae. More particularly, due to the wedge-like arrangement and a rigidity of the guide fingers 98, 100 and the implant keeper 28, the guide fingers 98, 100 force the opposing vertebrae apart from one another in a region of the annulus opening. Further, as the implant keeper 28 nears and enters the annulus opening, the implant keeper 28 serves to maintain the vertebral distraction due to a rigidity of the implant keeper 28.

The grip member 150 (FIG. 8) can then be actuated to distally eject the implant 22 from the implant keeper 28 into the intervertebral space. The guide fingers 98, 100 act to allow the implant 22 to smoothly pass between the vertebrae and in some embodiments, through the annulus hole into the intervertebral space. In particular, the guide fingers 98, 100 are deflected outwardly from the central longitudinal axis X as the implant 22 passes between them. The guide fingers 98, 100, in turn, abut against the top and bottom vertebra to provide better access to the intervertebral disc space. In one embodiment, the adjacent vertebrae are distracted apart as the implant 22 passes between the guide fingers 98, 100. Regardless, the smooth shape and texture of the guide fingers 98, 100 reduce friction between the implant 22 and two vertebrae, which can otherwise cause damage to either the vertebrae and/or the implant 22.

With the implant 22 in the desired position, the implant 22 is ejected from the guide piece 26 and remains in the intervertebral space. Unlike previous designs, the device 20 is configured in some embodiments such that a direction of travel of the implant 22 is generally parallel to the longitudinal axis X. Regardless, the top vertebra and the bottom vertebra can exert some force on the implant 22 such that the implant 22 is frictionally retained by the vertebrae and thereby removed from the guide piece 26 and/or the guide fingers 98, 100. Thus, with subsequent retraction of the guide finger 98, 100 from the target site, the implant 22 is not longer connected to the guide piece 26 and remains in the disc space.

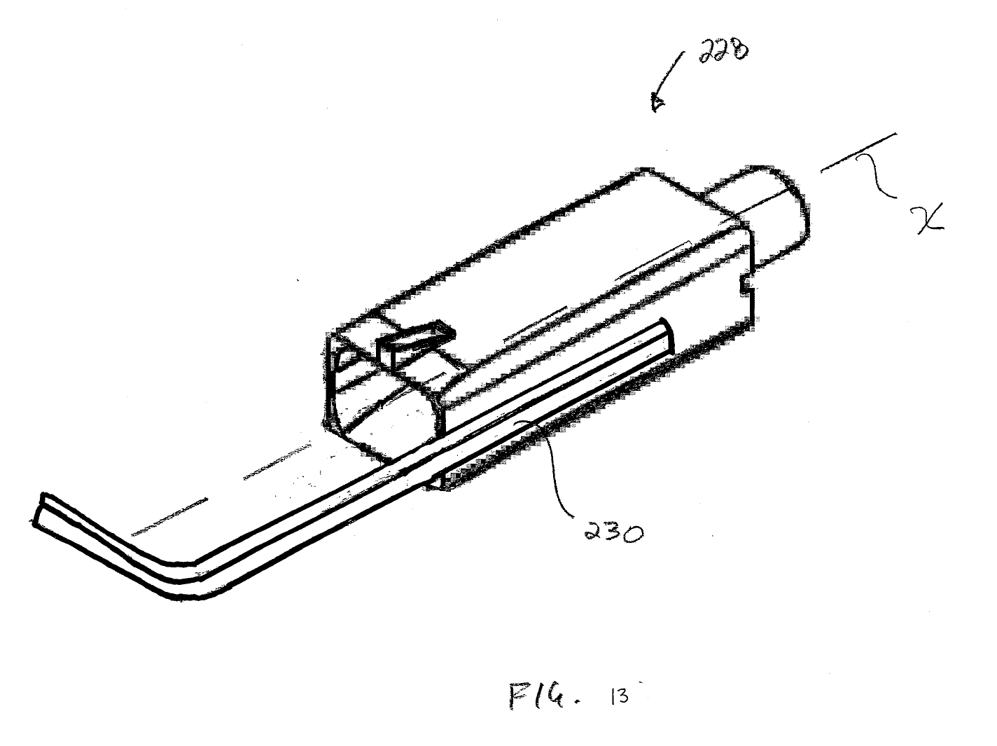

Another embodiment implant keeper 228 is shown in FIG. 13. The implant keeper 228 can be described as being substantially similar to the implant keeper 28 (FIG. 2). In addition, an end bracket 230 is provided. The end bracket 230 is permanently attached to implant keeper 228 or configured to be removable from the implant keeper 228 and/or interchangeable with other end brackets (not shown). In general terms, the end bracket 230 can act to significantly rotate the implant 22 (FIG. 2) up to approximately 90 degrees from where the implant 22 enters the disc space during insertion. The end bracket 230 forms a plurality of preformed curvatures or incorporates predetermined curvatures following insertion into disc space, for example. In one embodiment, the implant 22 is moved distally against the end bracket 230 by actuating the handle assembly 36 (FIG. 2) as previously described. The implant 22 is then guided by the end bracket through a 90 degree turn. In this manner, a user maneuvers the implant 22 into a position that is substantially orthogonal to the central longitudinal axis X.

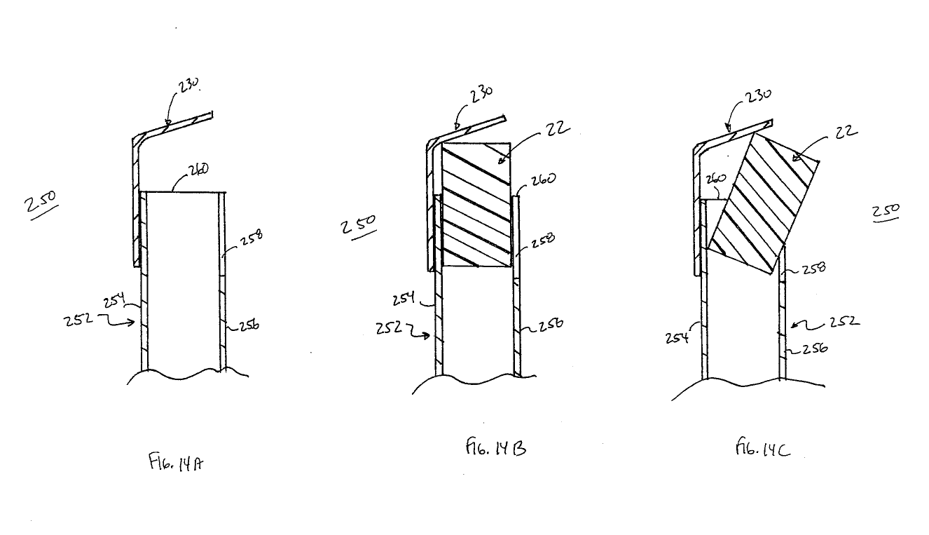

A distal portion of another, related alternative embodiment insertion device 250 is illustrated in simplified, cross-sectional form in FIG. 14A. The device 250 includes an implant keeper 252 akin to the implant keeper 28 (FIG. 2) previously described, along with the bracket 230. With the embodiment of FIG. 14A, the bracket 230 is attached to, and extends from, a first side wall 254 of the keeper 252. The keeper 252 further includes a second side wall 256 opposite the first side wall 254. With these conventions in mind, the second side wall 256 forms a slot 258 extending from, and open to, a distal end 260 of the keeper 252.

As shown in FIGS. 14B and 14C, the slot 258 facilitates or allows for a desired turning motion of the implant 22 as the implant is ejected from the keeper 252. For example, in FIG. 14B, the implant 22 has been distally moved into contact with the bracket 230. As a distal, axial force is further placed on the implant 22, interface with the bracket 230 causes the implant to “turn” (clockwise relative to FIG. 14B). In this regard, and as shown in FIG. 14C, the slot 258 provides for clearance of the implant 22 relative to the second side wall 256 in connection with this turning motion.

As a point of reference, while the implant 22 can take a variety of forms, one embodiment of the spinal implant 22 (e.g., a prosthetic spinal disc nucleus) includes an outer jacket (not shown) surrounding an expandable core (not shown). The spinal implant 22 narrows at a distal portion and at a proximal portion from a main body portion. The expandable core is formed of a hydrogel material, which upon hydration, expands to, and is constrained by, the outer jacket. Exemplary hydrogel core implants in accordance with the present invention are described in U.S. Pat. Nos. 5,824,093 and 6,132,465, the teachings of which are incorporated herein by reference. However, it should be understood that other spinal implants, including other types of hydrogel core implants or implants using springs or other mechanical means of supporting the intervertebral disc space are also contemplated. Thus, the insertion device 20 (FIG. 1) is in no way limited to any one particular spinal implant configuration or spinal surgical procedure, including both fusion and non-fusion procedures.

The device and method embodiments described above demonstrate that aspects of the present invention can achieve various advantages in spinal implant insertion procedures. For example, embodiments of the guide fingers 98, 100 (FIG. 2) help ensure reduced friction and smooth delivery of the implant 22 through the annulus and between adjacent vertebrae. In some embodiments, the guide fingers 98, 100, as well as the implant keeper 28, assist in distraction (or maintaining distraction) of the vertebrae at the intervertebral space, thus minimizing a force required to effectuate implantation. To this end, the guide finger 98, 100 effectively serve as a wedge in effectuating and maintaining vertebral distraction. Embodiments of the implant keeper 28 (FIG. 2) can also act to assist in maintaining a desired shape of the implant 22 in a hydrated, partially hydrated, or dehydrated state. Additionally, the screw mechanism used to introduce the implant 22 into the intervertebral space can help ensure a controlled and precise delivery of the implant 22. Other advantages of present invention, including the embodiments described herein, should be apparent to those having ordinary skill in the art upon viewing this specification and the accompanying figures. Additionally, one or more of the various components/assemblies described herein can be modified or replaced by an entirely different configuration and still meet the scope of the present invention.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. An insertion device for implanting a spinal implant in an intervertebral disc space, the insertion device comprising:
a guide piece configured to releasably receive a spinal implant;
an implant keeper releasably receiving the guide piece;
a guide structure extending distal the implant keeper; and
a handle assembly configured to effectuate distal movement of the guide piece relative to the implant keeper.

2. The device of claim 1, wherein the device further includes a spinal implant, and further wherein the implant keeper defines a cavity having a shape corresponding with a shape of the spinal implant, the cavity being exteriorly open at at least one side thereof for receiving the spinal implant.

3. The device of claim 2, wherein the cavity is cylindrical.

4. The device of claim 2, wherein the cavity is box-shaped.

5. The device of claim 1, wherein the implant keeper is configured to selectively retain at least a portion of the spinal implant.

6. The device of claim 1, wherein the implant keeper is rigid.

7. The device of claim 1, further comprising first and second protrusions extending outwardly from the implant keeper adjacent a distal end thereof.

8. The device of claim 7, wherein the protrusions extend in an opposing fashion from the implant keeper.

9. The device of claim 7, wherein the protrusions are proximally spaced from a distal end of the implant keeper.

10. The device of claim 7, wherein the guide structure is configured to slidably receive the protrusions.

11. The device of claim 1, wherein the guide structure includes first and second guide fingers extending distally from opposite sides of the implant keeper.

12. The device of claim 11, wherein the guide fingers are deflectable relative to one another opposite the implant keeper.

13. The device of claim 11, wherein each of the guide fingers is a metal tape.

14. The device of claim 11, wherein the guide fingers combine to define a wedge-like arrangement distal the implant keeper.

15. The device of claim 11, wherein the guide fingers are configured to effectuate vertebral distraction upon advancement of the guide fingers into an intradiscal space and in response to an expansion force generated by a spinal implant passing therebetween.

16. The device of claim 11, wherein an interior face of each of the guide fingers is smooth.

17. The device of claim 1, further comprising a bracket extending distal the implant keeper for causing movement of a spinal implant in a direction differing from a central longitudinal axis of the device.

18. The device of claim 17, wherein the bracket is removably attached to the implant keeper.

19. The device of claim 1, wherein the handle assembly is configured to translate a rotational input force into an axial movement of the guide piece.

20. The device of claim 1, further comprising:
a push rod connecting the guide piece and the handle assembly.

21. The device of claim 20, wherein the handle assembly distally actuates the push rod via a screw mechanism.

22. An insertion device for implanting a spinal implant into an intervertebral disc space, the device comprising:
a guide piece configured to releasably receiving a spinal implant;
an implant keeper defining an inner cavity, wherein the guide piece is slidably disposed within the cavity of the implant keeper;
a transition assembly including a pair of guide fingers and a base portion having an inner lumen, wherein the guide fingers extend along, and distally from, opposing sides of the implant keeper;
an insertion shaft defining an elongate tubular body having an inner lumen extending from a proximal end to a distal end, wherein the base portion includes threads;
a push rod defining a proximal end and a distal end, the push rod coaxially received in the inner lumen of the insertion shaft and the inner lumen of the base portion of the transition assembly; and
a handle assembly for distally advancing the push rod, the handle including,
a grip member having a threaded portion configured to mate with the threads of the insertion shaft.

23. A method of inserting a spinal implant into an intervertebral disc space through an annulus, the method comprising:
providing an insertion device including,
a guide piece configured to releasably receive a spinal implant,
an implant keeper releasably receiving the guide piece,
a guide structure extending distal the implant keeper,
a handle assembly configured to effectuate distal movement of the guide piece relative to the implant keeper;
forming an opening in a disc annulus;
removing a portion of a disc nucleus;
inserting at least a portion of the guide structure into the opening;
actuating the handle assembly; and
delivering the implant along the guide structure and into the intervertebral space.

24. The method of claim 23, wherein the guide structure includes first and second guide fingers extending distal the implant keeper, the method further comprising:
causing the guide fingers to effectuate distraction of opposing vertebrae associated with the disc space.

25. The method of claim 24, wherein the guide fingers form a wedge-like arrangement distal the implant keeper, whereby causing the guide fingers to effectuate distraction of opposing vertebrae includes distally advancing the guide fingers through the opening.

26. The method of claim 25, wherein distal advancement includes at least a portion of the implant keeper being positioned with the opening, the method further comprising the implant keeper maintaining an extent of distraction of the opposing vertebrae.

27. The method of claim 23, wherein causing the guide fingers to effectuate distraction includes:
translating an expansion force applied to the guide fingers by the spinal implant onto the opposing vertebrae.

28. The method of claim 23, wherein actuating the handle assembly includes:
rotating a grip portion of the assembly; and
translating rotation of the grip portion into longitudinal movement of the guide piece.
